(19) United States
(12) Patent Application Publication
(10) Pub. No.: US 2023/0375550 A1
(43) Pub. Date: Nov. 23, 2023

(54) Method for diagnosing breast cancer by using biomarker

(71) Applicant: Hangzhou CancerProbe Biological Technology Co., Ltd, Hangzhou (CN)

(72) Inventors: Supeng SUN, Hangzhou (CN); Xingyu ZHOU, Hangzhou (CN); Xiaonan KUI, Hangzhou (CN); Panpan YANG, Hangzhou (CN); Jing ZHOU, Hangzhou (CN); Liping SUN, Hangzhou (CN)

(21) Appl. No.: 18/315,213

(22) Filed: May 10, 2023

(30) Foreign Application Priority Data
May 18, 2022 (CN) 2022105479572

(51) Int. Cl. G01N 33/574; G01N 33/564

(52) U.S. Cl. CPC G01N 33/57415; G01N 33/564

(57) ABSTRACT

The present disclosure provides a biomarker for a breast cancer. The biomarker is a autoantibody combination. The biomarker comprises at least three autoantibodies against the following tumor-associated antigens: PARP1, TRIM21, P53, BRCA2, Annexin 11, ATAD2, NY-ESO-1, and CAGE. Through detection of the biomarker, an early screening for a breast cancer, particularly a triple-negative breast cancer, may be achieved. The present disclosure further provides an antigen protein combination for detecting the biomarker, a kit containing the antigen protein combination, and a corresponding detection or diagnosis method.

### CROSS-REFERENCE TO RELATED APPLICATIONS

The present disclosure claims the priority of the prior Chinese application No. 2022105479572, filed on May 18, 2022. All the disclosure of the present application including the description, claims, abstract, and abstract drawing is used in its entirety by the present disclosure.

### BACKGROUND OF THE INVENTION

### Field of the Invention

The present disclosure relates to the fields of biotechnology and medical diagnosis, in particular to an autoantibody biomarker for a breast cancer, an antigen combination for detecting the autoantibody biomarker, and use thereof in detecting a breast cancer.

### Description of the Related Art

Worldwide, breast cancer has become a leading cause of cancer-related deaths in women, the morbidity and mortality of which in women far exceed those of other cancers. In China, women over 50 years old have a high risk of breast cancer. The number of deaths due to breast cancer in this age group accounts for over 80% of all age groups. Among the breast cancer, triple-negative breast cancer (TNBC) is a subtype of breast cancer, which is characterized by lacked expressions of estrogen receptor (ER), progesterone receptor (PR), and human epidermal growth factor receptor 2 (HER2) in tumor tissues. This subtype of breast cancer is considered one of the most malignant types of breast cancer, accounting for 12-20% of all breast cancers, and has a higher risk of mortality due to its strong drug resistance, high recurrence rate, strong metastasis, and lack of targeted therapies.

Currently, the most urgent need is to improve the ability to detect the presence of early breast cancer, especially triple-negative breast cancer, thereby reducing the threat of the disease to humans and improving the survival rate and quality of life of a patient.

Breast cancer is usually detected using imaging techniques such as mammography, ultrasonic examination, and breast MRI examination. However, the detection results achieved with these techniques are related to the experience and technical quality of the physicians, and these techniques have defects of low resolution, and no guarantee of specificity and sensitivity. Therefore, it is necessary to develop more accurate, simple, low-risk, and non-invasive technical means to supplement or replace the conventional detection methods. In addition, tumor antigen markers have been proposed for screening breast cancer, such as human epidermal growth factor receptor 2 (HER2) antigen and carbohydrate antigen 15-3 (CA15-3). However, it is difficult to demonstrate that these markers are specific to breast cancer. Besides, the markers are limited to a tumor burden, and therefore have less value in early screening. Moreover, it is further proposed that gene markers such as BRCA1 and BRCA2, extracellular circulating DNAs (cell-free DNAs, cfDNAs), microRNAs (miRNAs) can be detected, but they do not have advantage for reducing the mortality of breast cancer.

An autoantibody refers to an antibody acting against tissues of the organism that produces it. In an early stage of carcinogenesis, an exposure to tumor-associated antigens could agitate human immune system, and then tumor-associated autoantibodies are produced and cascaded. A high level of the autoantibodies are maintained in the peripheral blood, which can be detected by a conventional biotechnological means in the art. It is universally acknowledged in the art that the autoantibodies induced by tumor antigens are a better index for early diagnosis of tumor. The autoantibodies generated by tumor induction could be used for reflecting disease progress of tumorigenesis of a patient, which is an important direction for searching a new target for early diagnosis and prognosis judgment of tumor.

Studies have proposed use of a single autoantibody or a combination of several autoantibodies for screening breast cancer. For example, the autoantibodies of p53, MUC1, HSP60, HSP90, HER2 and the like, and a joint detection of multiple autoantibodies proved to have improved sensitivity and specificity.

However, experiments indicated that the proposed autoantibody combination still has the defects in sensitivity of detecting breast cancer, particularly early-stage breast cancer. It is still necessary to find a new biomarker, such as an autoantibody combination related to a breast cancer specific antigen, for early screening and assisting diagnosis of breast cancer. Besides, there is also a need to provide a new autoantibody combination that is more sensitive for screening triple-negative breast cancer.

### BRIEF SUMMARY OF THE INVENTION

In order to solve the technical problems, the present disclosure finally identifies a group of autoantibodies for screening breast cancer, particularly in an early stage, by detecting autoantibodies aiming at different antigen targets in blood of a patient with a breast cancer. The autoantibody combination is used as a biomarker, and has a sufficiently high sensitivity particularly in detection of an early-stage tumor, particularly in an experimental Chinese population, and also has a sufficiently high detection specificity.

Therefore, an object of the present disclosure is to provide a biomarker for breast cancer which is an autoantibody combination.

Based on the autoantibody combination as a biomarker, another object of the present disclosure is to provide a reagent for detecting the autoantibody combination, for example, an antigen protein combination; and provide use of the autoantibody combination or the detection reagent in preparing a product for predicting a risk, performing screening, evaluating prognosis, monitoring a treatment effect or relapse and the like of a breast cancer.

Another object of the present disclosure to provide a kit and a corresponding method used in predicting a risk, performing screening, evaluating prognosis, monitoring a treatment effect or relapse and the like of a breast cancer.

The technical solution of the present disclosure is as follows.

In one aspect, the present disclosure provides a biomarker for a breast cancer. The biomarker is an autoantibody combination respectively comprising at least three of autoantibodies (tumor associated autoantibody, TAAb) of tumor-associated antigens (TAA) as follows: PARP1, TRIM21, P53, BRCA2, Annexin 11, ATAD2, NY-ESO-1, and CAGE.

In the context of the present disclosure, the accession numbers of the “tumor-associated antigens” in the UniProt database are as follows:
PARP1: P09874
TRIM21: P19474
P53: P04637
BRCA2: P51587
Annexin 11: P50995
ATAD2: Q6PL18
NY-ESO-1: P78358
CAGE: Q8TC20

Preferably, the autoantibody combination comprises autoantibodies respectively against the following tumor-associated antigens: PARP1, TRIM21, and CAGE.

More preferably, the autoantibody combination further comprises autoantibodies respectively against at least one, at least two, at least three, at least four, or at least five of the following tumor-associated antigens: P53, BRCA2, Annexin 11, ATAD2, and NY-ESO-1. The autoantibody combination preferably comprises autoantibodies against at least one, at least two, at least three, at least four, or at least four of the following tumor-associated antigens: Annexin 11, ATAD2, NY-ESO-1, and P53.

According to the specific embodiments of the present disclosure, the autoantibody combination comprises autoantibodies respectively against the following tumor-associated antigens:
(1) PARP1, TRIM21, and CAGE;
(2) PARP1, TRIM21, CAGE, and P53;
(3) PARP1, TRIM21, CAGE, and Annexin 11;
(4) PARP1, TRIM21, CAGE, and ATAD2;
(5) PARP1, TRIM21, CAGE, and NY-ESO-1;
(6) PARP1, TRIM21, CAGE, and BRCA2;
(7) PARP1, TRIM21, CAGE, Annexin 11, and ATAD2;
(8) PARP1, TRIM21, CAGE, Annexin 11, and NY-ESO-1;
(9) PARP1, TRIM21, CAGE, Annexin 11, and P53;
(10) PARP1, TRIM21, CAGE, ATAD2, and NY-ESO-1;
(11) PARP1, TRIM21, CAGE, ATAD2, and P53;
(12) PARP1, TRIM21, CAGE, NY-ESO-1, and P53;
(13) PARP1, TRIM21, CAGE, Annexin 11, ATAD2, and NY-ESO-1;
(14) PARP1, TRIM21, CAGE, Annexin 11, NY-ESO-1, and P53;
(15) PARP1, TRIM21, CAGE, ATAD2, NY-ESO-1, and P53; and
(16) PARP1, TRIM21, P53, BRCA2, Annexin 11, ATAD2, NY-ESO-1, and CAGE.

According to the present disclosure, the autoantibody is an autoantibody in a sample of an individual, such as whole blood, serum, plasma, a tissue or a cell, interstitial fluid, cerebrospinal fluid, or urine, wherein preferably, the tissue or the cell is a breast tissue or cell, a breast cancer tissue or cell, or a para-carcinoma tissue or cell of a breast cancer.

Preferably, the individual is a mammal, preferably, a primate mammal, more preferably, human

Preferably, the autoantibody is IgA (e.g. IgA1 and IgA2), IgM, or IgG (e.g. IgG1, IgG2, IgG3, and IgG4).

Preferably, the breast cancer comprises a ductal carcinoma in situ and an invasive breast cancer, wherein the invasive breast cancer comprises an invasive ductal carcinoma, an invasive micropapillary carcinoma, an invasive lobular carcinoma, and a mucosecretory carcinoma. Based on TNM staging, the breast cancer is a stage I or II breast cancer.

Preferably, the breast cancer comprises a triple-negative breast cancer and a non-triple-negative breast cancer, especially a triple-negative breast cancer.

According to the present disclosure, the biomarker, an autoantibody combination, may be detected in the sample of the individual (e.g. plasma or serum). In the present disclosure, “presence” or “absence” and “positive” or “negative” of an autoantibody are used interchangeably. It is a conventional technology in the art to determine from this. For example, detection may be performed by an antigen-antibody specific reaction between tumor-associated antigens appearing due to any autoantibody in the combination and the autoantibodies. Therefore, correspondingly, in another aspect, the present disclosure further provides a reagent for detecting a biomarker of the present disclosure.

Depending on specific technological means, the reagent may be used in an enzyme-linked immunosorbent assay (ELISA), a protein/peptide fragment chip detection, an immunoblotting, a microbead immunoassay or a microfluidic immunoassay, etc. Preferably, the reagent is used for detecting the biomarker of the present disclosure by an antigen-antibody reaction, for example, by ELISA or fluorescence or chemiluminescence immunoassay.

In the aspect, the reagent may be an antigen protein combination comprising at least three of the following tumor-associated antigens selected from: PARP1, TRIM21, P53, BRCA2, Annexin 11, ATAD2, NY-ESO-1, and CAGE.

Preferably, the antigen protein combination comprises following tumor-associated antigens: PARP1, TRIM21, and CAGE.

More preferably, the antigen protein combination further comprises at least one, at least two, at least three, at least four, or at least five of the following tumor-associated antigens: P53, BRCA2, Annexin 11, ATAD2, and NY-ESO-1. The antigen protein combination preferably comprises at least one, at least two, at least three, or at least four of the following tumor-associated antigens: Annexin 11, ATAD2, NY-ESO-1, and P53.

Further preferably, the antigen protein combination comprises following tumor-associated antigens:
(1) PARP1, TRIM21, and CAGE;
(2) PARP1, TRIM21, CAGE, and P53;
(3) PARP1, TRIM21, CAGE, and Annexin 11;
(4) PARP1, TRIM21, CAGE, and ATAD2;
(5) PARP1, TRIM21, CAGE, and NY-ESO-1;
(6) PARP1, TRIM21, CAGE, and BRCA2;
(7) PARP1, TRIM21, CAGE, Annexin 11, and ATAD2;
(8) PARP1, TRIM21, CAGE, Annexin 11, and NY-ESO-1;
(9) PARP1, TRIM21, CAGE, Annexin 11, and P53;
(10) PARP1, TRIM21, CAGE, ATAD2, and NY-ESO-1;
(11) PARP1, TRIM21, CAGE, ATAD2, and P53;
(12) PARP1, TRIM21, CAGE, NY-ESO-1, and P53;
(13) PARP1, TRIM21, CAGE, Annexin 11, ATAD2, and NY-ESO-1;
(14) PARP1, TRIM21, CAGE, Annexin 11, NY-ESO-1, and P53;
(15) PARP1, TRIM21, CAGE, ATAD2, NY-ESO-1, and P53; and
(16) PARP1, TRIM21, P53, BRCA2, Annexin 11, ATAD2, NY-ESO-1, and CAGE.

In other aspect, the present disclosure provides use of the biomarker or the reagent in preparing a product for predicting a risk, performing screening, evaluating prognosis, monitoring a treatment effect or relapse and the like of a breast cancer.

Preferably, the breast cancer comprises a ductal carcinoma in situ and an invasive breast cancer, wherein the invasive breast cancer comprises an invasive ductal carcinoma, an invasive micropapillary carcinoma, an invasive lobular carcinoma, and a mucosecretory carcinoma. Based on TNM staging, the breast cancer is a stage I or II breast cancer.

Preferably, the breast cancer comprises a triple-negative breast cancer and a non-triple-negative breast cancer, especially a triple-negative breast cancer.

Preferably, the product is a kit; more preferably, the kit is used for an enzyme-linked immunosorbent assay (ELISA), a protein/peptide fragment chip detection, an immunoblotting, a microbead immunoassay or a microfluidic immunoassay; and preferably, the kit is used for detecting the biomarker by an antigen-antibody reaction, for example, by an ELISA kit or a fluorescence or chemiluminescence immunoassay kit.

In another aspect, the present disclosure provides a kit comprising the reagent of the present disclosure.

Depending on specific technological means, the kit may be used in an enzyme-linked immunosorbent assay (ELISA), a protein/peptide fragment chip detection, an immunoblotting, a microbead immunoassay or a microfluidic immunoassay, etc. Preferably, the kit is used for detecting the biomarker of the present disclosure by an antigen-antibody reaction, for example, by an ELISA kit or a fluorescence or chemiluminescence immunoassay kit.

Therefore, preferably, the kit is an enzyme-linked immunosorbent assay (ELISA) kit. That is, the kit is used to detect whether an autoantibody biomarker in the sample of the individual is positive by an enzyme-linked immunosorbent assay. Accordingly, the kit may further comprise other components necessary for ELISA detection of the autoantibody biomarker, all well-known in the art. For a purpose of detection, for example, an antigen protein in the kit may be linked to a tag peptide, such as a His tag, a streptavidin tag, and a Myc tag; for another example, the kit may comprise a solid-phase carrier, for example, a microwell carrier to which an antigen protein may be immobilized, for example, an ELISA plate; or a microbead or magnetic-bead solid-phase carrier. The kit may further comprise an adsorption protein for immobilizing an antigen protein on a solid-phase carrier, a diluent for blood such as serum, a washing solution, an enzyme-labeled secondary antibody or a fluorescent or chemiluminescent substance, a color developing solution, a stopping solution, etc. A concentration of the corresponding antibody in a body fluid is detected by a principle that an antigen protein indirectly or directly coated on a surface of the solid-phase carrier reacts with an antibody in serum/plasma/interstitial fluid and the like to form an antigen-antibody complex.

In yet another aspect, the present disclosure provides a method for predicting a risk, performing screening, evaluating prognosis, monitoring a treatment effect or relapse and the like of a breast cancer. The method comprises the following steps:
(1) quantifying each autoantibody in the autoantibody combination provided by the present disclosure on a sample from an individual; and
(2) comparing the amount of the autoantibody to a reference cutoff value, and when the amount is above the reference cutoff value, determining that the individual has a risk of developing a breast cancer, suffers from a breast cancer, has a poor prognosis and a poor treatment effect for a breast cancer, or has a risk of relapse.

In step (1), the quantification comprises detecting each autoantibody in the autoantibody combination by using the reagent (i.e., an antigen protein combination) provided by the present disclosure or a kit containing the reagent.

According to the present disclosure, the individual is a mammal, preferably, a primate mammal, more preferably, human.

Preferably, the breast cancer comprises a ductal carcinoma in situ and an invasive breast cancer, wherein the invasive breast cancer comprises an invasive ductal carcinoma, an invasive micropapillary carcinoma, an invasive lobular carcinoma, and a mucosecretory carcinoma. Based on TNM staging, the breast cancer is a stage I or II breast cancer.

Preferably, the breast cancer comprises a triple-negative breast cancer and a non-triple-negative breast cancer, especially a triple-negative breast cancer.

According to the present disclosure, the sample is a sample of an individual, such as whole blood, serum, plasma, a tissue or a cell, interstitial fluid, cerebrospinal fluid, or urine, wherein preferably, the tissue or the cell is a breast tissue or cell, a breast cancer tissue or cell, or a para-carcinoma tissue or cell of a breast cancer.

In step (2), the reference cutoff value may be a reference level from healthy subjects or a healthy population, for example, the reference cutoff value may be defined as the mean plus 2 standard deviations of a population confirmed not suffering from a cancer by a physical examination.

Compared with the prior art, the present disclosure provides a novel biomarker which is a whole-new group of tumor autoantibodies and related to a breast cancer, particularly a triple-negative breast cancer. These autoantibodies have a higher positive detection rate in triple-negative breast cancer and also have an independent positive contribution rate. When in a combined use, the autoantibody combination of the present disclosure has a very good detection sensitivity and a high enough detection specificity, and may have a sensitivity of 60% or more of a triple-negative breast cancer.

In addition, the biomarker provided by the present disclosure may be detected in a serum sample of an individual, is used for detecting a breast cancer, especially a triple-negative breast cancer with a high malignancy, and assists in clinical diagnosis of the disease. The clinical diagnosis of the breast cancer is assisted by a serum detection, such that more patients with early-stage triple-negative breast cancer may be found, the life cycle of the patients is prolonged, the quality of life is improved, and the biomarker has a wide application prospect.

In another aspect, the present disclosure provides a method for detecting whether an individual suffers from a breast cancer, comprising the following steps: providing a biological sample for an individual; and detecting an autoantibody of an antigen protein in the biological sample to obtain a quantitative value of the autoantibody, wherein if the quantitative value of the autoantibody exceeds or equals to a cutoff value, the individual is determined to be a patient with a breast cancer; if the quantitative value of the autoantibody is less than the cutoff value, the individual is determined not to be a patient with a breast cancer; and the antigen is selected from at least one or more than one antigen combination of the following antigens: PARP1, TRIM21, P53, BRCA2, Annexin 11, ATAD2, NY-ESO-1, and CAGE.

In some embodiments, the antigen is PARP1, TRIM21, and CAGE.

In some embodiments, the antigen is selected from one of the antigen combination:
(1) PARP1, TRIM21, and CAGE;
(2) PARP1, TRIM21, CAGE, and P53;
(3) PARP1, TRIM21, CAGE, and Annexin 11;
(4) PARP1, TRIM21, CAGE, and ATAD2;
(5) PARP1, TRIM21, CAGE, and NY-ESO-1;
(6) PARP1, TRIM21, CAGE, and BRCA2;
(7) PARP1, TRIM21, CAGE, Annexin 11, and ATAD2;
(8) PARP1, TRIM21, CAGE, Annexin 11, and NY-ESO-1;
(9) PARP1, TRIM21, CAGE, Annexin 11, and P53;
(10) PARP1, TRIM21, CAGE, ATAD2, and NY-ESO-1;
(11) PARP1, TRIM21, CAGE, ATAD2, and P53;
(12) PARP1, TRIM21, CAGE, NY-ESO-1, and P53;
(13) PARP1, TRIM21, CAGE, Annexin 11, ATAD2, and NY-ESO-1;
(14) PARP1, TRIM21, CAGE, Annexin 11, NY-ESO-1, and P53;
(15) PARP1, TRIM21, CAGE, ATAD2, NY-ESO-1, and P53; and
(16) PARP1, TRIM21, P53, BRCA2, Annexin 11, ATAD2, NY-ESO-1, and CAGE.

In some embodiments, the sample of the individual is selected from one or more of the following samples: whole blood, serum, plasma, a tissue, a cell, interstitial fluid, cerebrospinal fluid, or urine.

In some embodiments, the sample is one of whole blood, serum, and plasma.

In some embodiments, the individual is a mammal.

In some embodiments, the autoantibody is IgA, IgM, or IgG.

In some embodiments, the breast cancer comprises a ductal carcinoma in situ and an invasive breast cancer; or a triple-negative breast cancer and a non-triple-negative breast cancer, or a stage I or II breast cancer.

In some embodiments, the detection comprises one of the following modes: an enzyme-linked immunosorbent assay (ELISA), a protein/peptide fragment chip detection, an immunoblotting, a microbead immunoassay or a microfluidic immunoassay.

In some embodiments, the absorbance cutoff value of the autoantibody of the antigen is as follows: Anti-PARP1 has an absorbance cutoff value of 1.5, Anti-TRIM21 has an absorbance cutoff value of 0.5, Anti-P53 has an absorbance cutoff value of 1.2, Anti-BRCA2 has an absorbance cutoff value of 1.0, Anti-Annexin 11 has an absorbance cutoff value of 1.0, Anti-ATAD2 has an absorbance cutoff value of 0.5, Anti-NY-ESO-1 has an absorbance cutoff value of 1.0, and Anti-CAGE has an absorbance cutoff value of 1.1.

In another aspect, the present disclosure provides a method for detecting whether an individual suffers from a triple-negative breast cancer, comprising the following steps:
providing a biological sample for an individual; and
detecting an autoantibody of an antigen protein in the biological sample to obtain a quantitative value of the autoantibody,
wherein if the quantitative value of the detected autoantibody of the antigen exceeds or equals to a cutoff value, the individual is determined to be a patient with a triple-negative breast cancer; if the quantitative value of the detected autoantibody of the antigen is less than the cutoff value, the individual is determined not to be a patient with a breast cancer; and the antigen is selected from at least one or more than one antigen of the following antigens: PARP1, TRIM21, P53, BRCA2, Annexin 11, ATAD2, NY-ESO-1, and CAGE.

In some embodiments, the antigen is PARP1, TRIM21, and CAGE.

In some embodiments, the antigen is one combination in the following group:
(1) PARP1, TRIM21, and CAGE;
(2) PARP1, TRIM21, CAGE, and P53;
(3) PARP1, TRIM21, CAGE, and Annexin 11;
(4) PARP1, TRIM21, CAGE, and ATAD2;
(5) PARP1, TRIM21, CAGE, and NY-ESO-1;
(6) PARP1, TRIM21, CAGE, and BRCA2;
(7) PARP1, TRIM21, CAGE, Annexin 11, and ATAD2;
(8) PARP1, TRIM21, CAGE, Annexin 11, and NY-ESO-1;
(9) PARP1, TRIM21, CAGE, Annexin 11, and P53;
(10) PARP1, TRIM21, CAGE, ATAD2, and NY-ESO-1;
(11) PARP1, TRIM21, CAGE, ATAD2, and P53;
(12) PARP1, TRIM21, CAGE, NY-ESO-1, and P53;
(13) PARP1, TRIM21, CAGE, Annexin 11, ATAD2, and NY-ESO-1;
(14) PARP1, TRIM21, CAGE, Annexin 11, NY-ESO-1, and P53;
(15) PARP1, TRIM21, CAGE, ATAD2, NY-ESO-1, and P53; and
(16) PARP1, TRIM21, P53, BRCA2, Annexin 11, ATAD2, NY-ESO-1, and CAGE.

In some embodiments, the sample of the individual is selected from one or more of the following samples: whole blood, serum, plasma, a tissue, a cell, interstitial fluid, cerebrospinal fluid, or urine. In some embodiments, the sample is whole blood, serum, and plasma samples.

In some embodiments, mode absorbance cutoff value of the autoantibody of the antigen is as follows: Anti-PARP1 has an absorbance cutoff value of 1.5, Anti-TRIM21 has an absorbance cutoff value of 0.5, Anti-P53 has an absorbance cutoff value of 1.2, Anti-BRCA2 has an absorbance cutoff value of 1.0, Anti-Annexin 11 has an absorbance cutoff value of 1.0, Anti-ATAD2 has an absorbance cutoff value of 0.5, Anti-NY-ESO-1 has an absorbance cutoff value of 1.0, and Anti-CAGE has an absorbance cutoff value of 1.1.

In another aspect, the present disclosure provides a method for detecting whether an individual suffers from a stage I or II breast cancer, comprising the following steps:
providing a biological sample for an individual; and
detecting an autoantibody of an antigen protein in the biological sample to obtain a quantitative value of the autoantibody,
wherein if the quantitative value of the detected autoantibody of the antigen exceeds or equals to a cutoff value, the individual is determined to be a patient with a stage I or II breast cancer; if the quantitative value of the detected autoantibody of the antigen is less than the cutoff value, the individual is determined not to be a patient with a breast cancer; and the antigen is selected from at least one or more than one antigen combination of the following antigens: PARP1, TRIM21, P53, BRCA2, Annexin 11, ATAD2, NY-ESO-1, and CAGE.

In some embodiments, the antigen is PARP1, TRIM21, and CAGE.

In some embodiments, the absorbance cutoff value of the autoantibody of the antigen is as follows: Anti-PARP1 has an absorbance cutoff value of 1.5, Anti-TRIM21 has an absorbance cutoff value of 0.5, Anti-P53 has an absorbance cutoff value of 1.2, Anti-BRCA2 has an absorbance cutoff value of 1.0, Anti-Annexin 11 has an absorbance cutoff value of 1.0, Anti-ATAD2 has an absorbance cutoff value of 0.5, Anti-NY-ESO-1 has an absorbance cutoff value of 1.0, and Anti-CAGE has an absorbance cutoff value of 1.1.

### BRIEF DESCRIPTION OF THE DRAWINGS

The technical solutions of the present disclosure are described in details below with reference to the accompanying drawings.

FIGS. 1-8 show a relative concentration of each tumor autoantibody in blood of shown populations (1: a healthy population through a physical examination; 2: a patient population with a triple-negative breast cancer; and 3: a patient population with a benign breast disease);

FIGS. 9-16 show a receiver operating characteristic curve (ROC curve) and an area under the curve (AUC) for each tumor autoantibody in distinguishing a triple-negative breast cancer;

FIG. 17 shows an ability of a 3-TAAb combination consisting of PARP1, TRIM21, and CAGE in detecting a triple-negative breast cancer;

FIG. 18 shows an ROC curve of a 8-TAAb combination consisting of 8 autoantibodies in a triple-negative breast cancer;

FIG. 19 shows an ROC curve of a 8-TAAB combination consisting of 8 autoantibodies in distinguishing a triple-negative breast cancer from a benign breast disease; and

FIG. 20 shows an ROC curve of a 8-TAAB combination consisting of 8 autoantibodies in distinguishing an early-stage breast cancer.

### DETAILED DESCRIPTION OF THE INVENTION

In the present disclosure, the term “antigen” or “antigen protein” is used interchangeably. In addition, the present disclosure relates to the following experimental operations or definitions. It should be noted that the present disclosure may also be implemented by other conventional technologies in the art, and is not limited to the following experimental operations.

(I) Preparation of Recombinant Antigen Protein

A cDNA fragment of a tumor antigen is cloned into an expression vector containing a 6×His-labeled PET28(a). At an N-terminal or a C-terminal of the antigen, streptavidin or an analogue thereof (biotin-binding tag protein) is introduced. The obtained recombinant expression vector is transformed into Escherichia coli for expression. A protein expressed in a supernatant is purified by a Ni-NTA affinity column and an ion column. When the protein is expressed in an inclusion body, the protein is denatured by 6 M guanidine hydrochloride, renatured and folded in vitro according to a standard method, and then purified by a Ni-NTA affinity column through a 6×His tag to obtain an antigen protein.

(II) Preparation and Preservation of Serum or Plasma

Serum or plasma of a patient with a breast cancer is collected when the patient is initially diagnosed with a breast cancer and has not received any radiotherapy, chemotherapy, or surgical treatment. The plasma or the serum is prepared according to a standard clinical procedure and stored in a refrigerator at −80° C. for a long period.

(III) ELISA Detection

A concentration of an autoantibody in the sample is quantified by an enzyme linked immunosorbent assay (ELISA).

The purified tumor antigen is immobilized to a surface of a microwell by a tag streptavidin or an analogue thereof. The microwell is pre-coated with a biotin-labeled bovine serum albumin (BSA). The serum or plasma sample is diluted at 1:110 with a phosphate buffer and added into a microwell for a reaction (50 ml/well). After an unbound serum or plasma component is washed with a washing solution, a horseradish peroxidase (HRP)-conjugated anti-human IgG is added per well for a reaction. Then a reaction substrate, 3,3′,5,5′-tetramethylbenzidine (TMB) is added to develop color. A stop solution (1 N HCl) is added and an absorbance (OD) is read by a microplate reader at a single spectrum of 450 nm. A serum autoantibody concentration is quantified using a standard curve.

(IV) Cutoff Value of Autoantibody

A cutoff value of an autoantibody is defined as the mean of a detected absorbance value plus 2 standard deviations (SD) or the mean plus 3 standard deviations (SD) of a control normal population confirmed not suffering from a cancer by a physical examination. The cutoff value of each autoantibody is determined according to the following principles: 1. in a case of using two different values (mean plus two standard deviations and mean plus three standard deviations) as a reference cutoff value, a detection specificity of each autoantibody to the control normal population is 95% or more; and 2. in a case of using the two different values as a reference cutoff value, a specificity of each autoantibody to the control normal population and a sensitivity to a patient population with a breast cancer are obtained, a sum of the two is calculated, and a value at a larger sum is selected as a determined cutoff value of the autoantibody.

(V) Positive and Negative Determination of Single Autoantibody

With determination of each autoantibody, after a level of the autoantibody in the sample is quantified and the level is compared with the cutoff value, a positive reaction is defined as positive when the level is more than or equal to the cutoff value; and accordingly, a negative reaction is defined as negative when the level less than the cutoff value. The cutoff (absorbance cutoff value) of the autoantibody of the antigen protein of the present invention is as follows: Anti-PARP1 has an absorbance cutoff value of 1.5, Anti-TRIM21 has an absorbance cutoff value of 0.5, Anti-P53 has an absorbance cutoff value of 1.2, Anti-BRCA2 has an absorbance cutoff value of 1.0, Anti-Annexin 11 has an absorbance cutoff value of 1.0, Anti-ATAD2 has an absorbance cutoff value of 0.5, Anti-NY-ESO-1 has an absorbance cutoff value of 1.0, and Anti-CAGE has an absorbance cutoff value of 1.1.

(VI) Positive Determination of Autoantibody Combination

Since a positive rate of a single autoantibody is low, in order to increase a detection positive rate of an autoantibody, when a result is analyzed, a predicted effect is determined in combination with a result of a plurality of autoantibodies. A rule is that when a plurality of autoantibodies are detected in a sample, as long as one or more autoantibodies are positive, the antibody combination result is determined to be positive; however, if all the autoantibodies are negative, the antibody combination result is determined to be negative.

(VII) Statistical Analysis Method

Two groups are statistically analyzed using a Mann-Whitney U test using GraphPad Prism v.6 (GraphPad Prism software, San Diego, California) and IBM SPSS Statistics 23 for Windows (IBM, New York). In analyzing a relationship between each parameter, a Spearman's correlation analysis is performed.

(VIII) Determination of Sensitivity and Specificity

Sensitivity: in all cases with diseases diagnosed by a gold standard, a proportion of cases with a positive detection result of an autoantibody or an autoantibody combination in all the cases with diseases.

Specificity: in all subjects diagnosed to be disease-free, a proportion of subjects with a negative detection result of an autoantibody or an autoantibody combination in all the subjects.

(IX) Clinical Evaluation Index

Expression levels of estrogen receptor (ER), progestin receptor (PR), and human epidermal growth factor receptor 2 (HER2) in a pathological report of a breast cancer are obtained according to a conventional immunohistochemical and medical standardized pathological report analysis. ER and PR positive are defined as equal to or more than 1% positively stained tumor cells.

In this study, ER expression levels are artificially grouped as:
ER (−): ER negative;
ER (medium): 1%-50% positively stained tumor cells; and
ER (high): >50% positively stained tumor cells.

PR expression levels are artificially grouped as:
PR (−): PR negative;
PR (medium): 1%-10% positively stained tumor cells; and
PR (high): >10% positively stained tumor cells.

An immunohistochemical examination of an expression amount of HER2 on a cell surface of a breast cancer tissue shows a result ranging from HER2(0) to HER2(3+). Over 10% of cells show a strong staining of an intact membrane to 3+. The expression amount is from 0 to 1+ and the expression amount of HER2 is considered negative. If the result is 2+, the expression amount is critical. In this case, an HER2 gene amplification is further detected by using an in situ hybridization method. When a single-copy HER2 gene is more than 6 or a HER2/CEP17 ratio is more than 2.0, the expression amount of HER2 is positive, otherwise, the expression amount is negative.

The present disclosure is described below with reference to the specific examples. It could be understood by a person skilled in the art that these examples are merely used for describing the present disclosure, but do not limit the protection scope of the present disclosure in any methods. A sample collection has been informed by a subject or a patient and approved by the regulatory authorities.

Experimental methods in the following examples are conventional, unless otherwise specified. The raw materials, the reagent materials, etc. used in the following examples are all commercially available, unless otherwise specified.

### Example 1

A discovery cohort comprising 90 healthy subjects by a physical examination, 92 patients with benign breast disease, and 40 patients with triple-negative breast cancer was involved into the establishment of a breast cancer biomarker. Serum samples from the patients with breast cancer were obtained from the Shanghai Cancer Center. The healthy subjects and the patients with benign breast disease were from 4 different medical centers. The serums of all the patients with breast cancer were collected when the patients were initially diagnosed as triple-negative breast cancer and had not received any radiotherapy, chemotherapy, or surgical treatment, and were stored at −80° C.

8 antigens (Table 1) were coated on the surface of 96-well plates and then the plates were blocked and reacted with the serums diluted at 1:110 of the patients with breast cancer, the healthy subjects, or the patients with benign breast disease, and then reacted with an anti-human IgG antibody-HRP horseradish peroxidase. After a color development reaction, the absorbance of the plates were read at a wavelength of 450 nm. Table 1 shows detection sensitivity and specificity of each autoantibody.

TABLE 1
Sensitivity and specificity of single tumor
autoantibody marker of discovery cohort

| Antigen | No. | Sensitivity | Specificity |
|---|---|---|---|
| PARP1 | P09874 | 40.0% | 92.1% |
| TRIM21 | P19474 | 47.5% | 90.0% |
| P53 | P04637 | 22.5% | 86.7% |
| BRCA2 | P51587 | 17.5% | 88.9% |
| Annexin 11 | P50995 | 32.5% | 87.6% |
| ATAD2 | Q6PL18 | 23.1% | 89.9% |
| NY-ESO-1 | P78358 | 35.0% | 90.0% |
| CAGE | Q8TC20 | 47.5% | 88.9% |

In the present disclosure, a plurality of control populations from different hospitals were used so as to more accurately represent settings of clinical application, thereby enabling the detection to be more clinically significant.

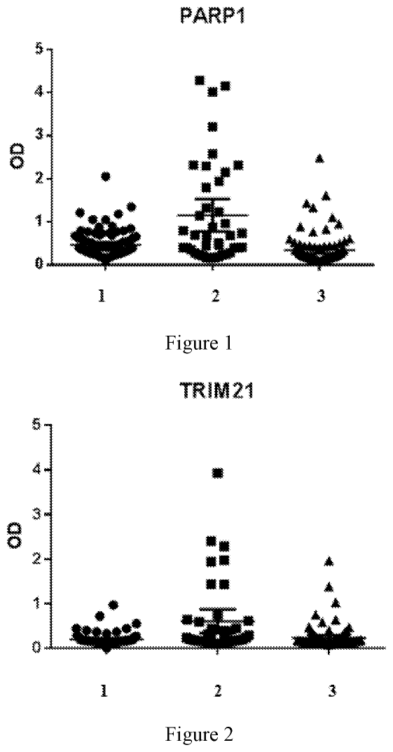

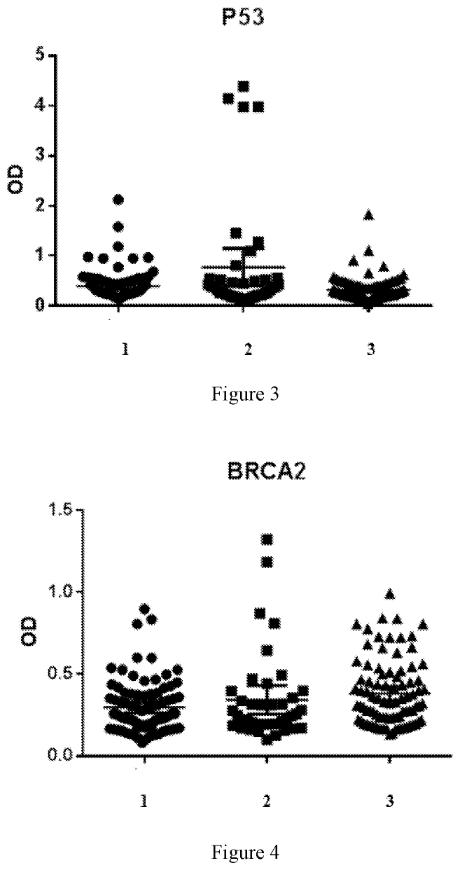

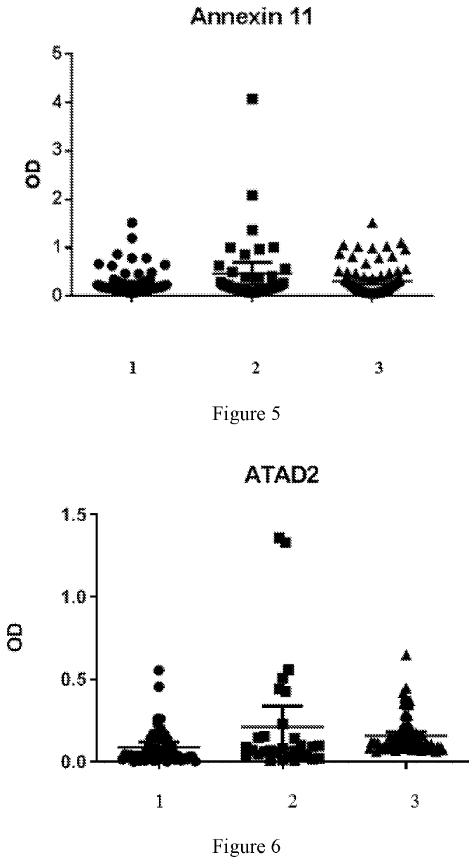

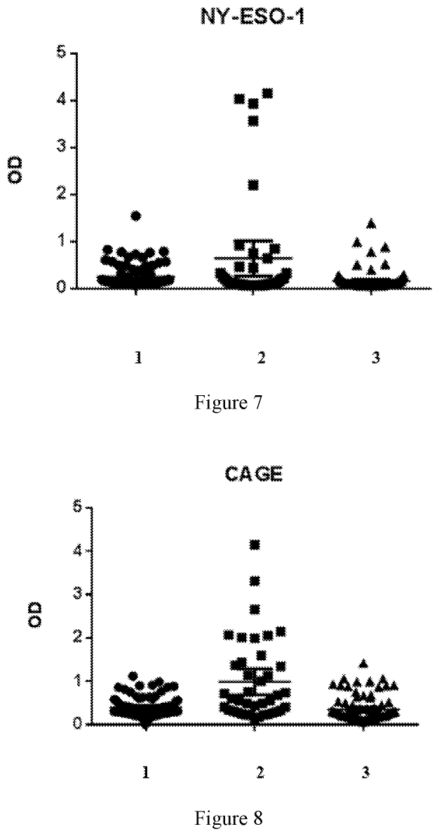

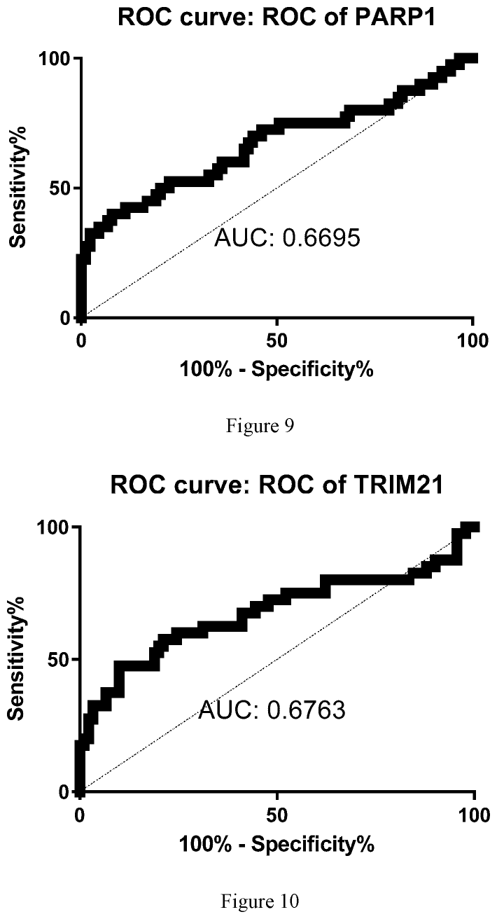

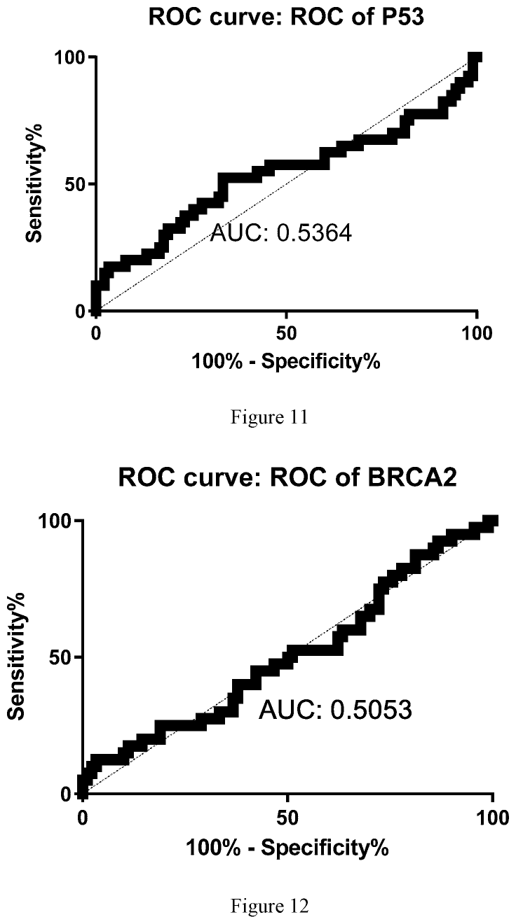

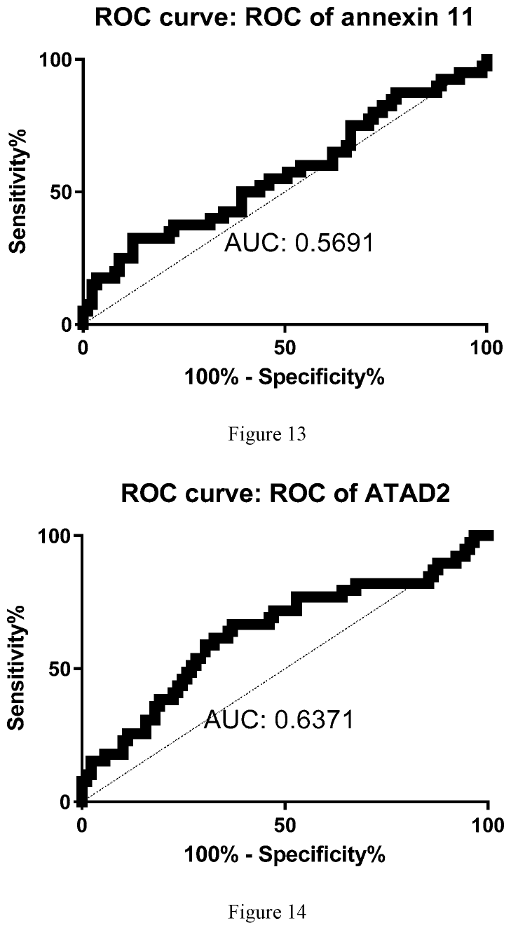

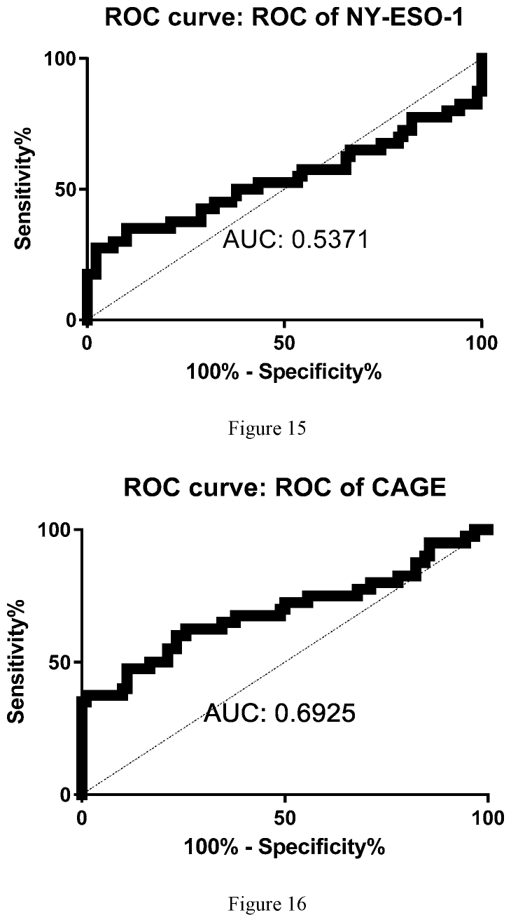

The detection results proved that each autoantibody had a contribution to improve the positive rates of the patients with triple-negative breast cancer. The sensitivity and the specificity of each antibody of the cohort (90 healthy subjects by a physical examination, 92 patients with benign breast disease, and 40 patients with triple-negative breast cancer) are shown in Table 1. The relative concentrations of each tumor autoantibody in different populations are shown in FIGS. 1-8. Receiver operating characteristic curve (ROC) and area under the curve (AUC) of each tumor autoantibody in distinguishing the triple-negative breast cancer are shown in FIGS. 9-16 and Table 2 respectively.

TABLE 2
AUC value of autoantibody of each antigen

| Antigen | No. | AUC value |
|---|---|---|
| PARP1 | P09874 | 0.6695 |
| TRIM21 | P19474 | 0.6763 |
| P53 | P04637 | 0.5364 |
| BRCA2 | P51587 | 0.5053 |
| Annexin11 | P50995 | 0.5691 |
| ATAD2 | Q6PL18 | 0.6371 |
| NY-ESO-1 | P78358 | 0.5371 |
| CAGE | Q8TC20 | 0.6925 |

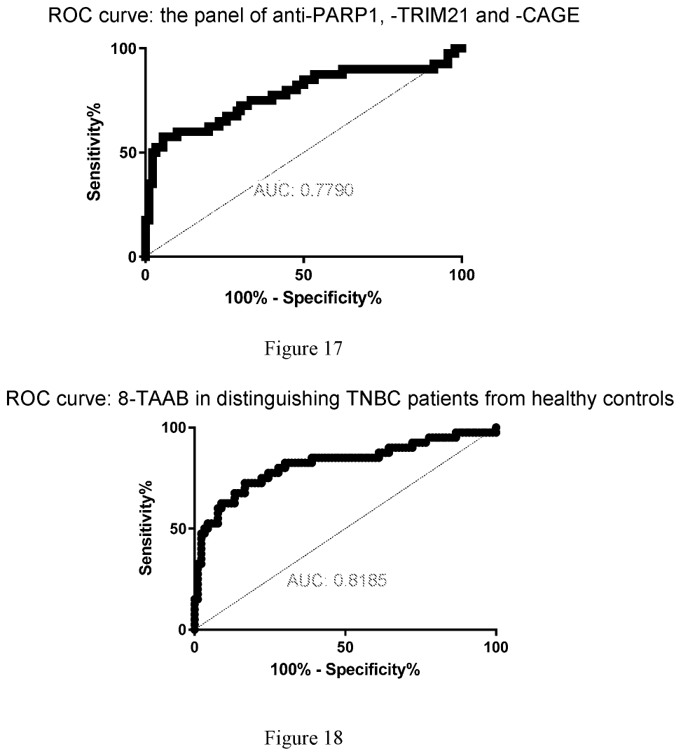

As a result, it can be known that three molecules, anti-PARP1, anti-TRIM21 and anti-CAGE, may be used as core molecules in the present disclosure. From Table 1, it can be seen that the sensitivity of these three molecules is the first three with similar specificities. The three molecules formed an autoantibody combination, whose sensitivity was 60.0%, and specificity was 90.0%. The AUC of the combination was 0.7790, 95% CI (0.6802, 0.8779), P<0.0001, as shown in FIG. 17. The addition of other molecules also contributed new cases of a triple-negative breast cancer, increasing the detection efficacy of the autoantibody combination.

The autoantibodies all had a higher specificity but a lower sensitivity, and the sensitivity of each autoantibody was between 17.5% and 47.5%. In the aspect of the detection efficacy (AUC), the AUC of single autoantibody was between 0.5053 and 0.6925. The combination of 8 autoantibodies had a sensitivity of 62.5%, a specificity of 91.1%, a detection efficacy AUC of 0.8185, 95% CI (0.7292, 0.9078), and P<0.0001 for triple-negative breast cancer as shown in FIG. 18,

Kruskal-Wallis test was employed for the scatter diagrams, which found that except anti-Annexin 11, the relative levels of other molecules in the patients with triple-negative breast cancer were significantly higher than the healthy subjects and the patients with benign breast disease (P≤0.05). The Kruskal-Wallis test results are shown in Table 3. Thus, the markers are important for a highly specific detection of triple-negative breast cancer.

TABLE 3
Statistical Kruskal-Wallis detection of single autoantibody marker
at level of patients with triple-negative breast cancer

| Antigen | No. | Kruskal-Wallis detection result (P value) |
|---|---|---|
| PARP1 | P09874 | <0.0001 |
| TRIM21 | P19474 | 0.0005 |
| P53 | P04637 | 0.0164 |
| BRCA2 | P51587 | 0.0113 |
| Annexin 11 | P50995 | 0.422 |
| ATAD2 | Q6PL18 | <0.0001 |
| NY-ESO-1 | P78358 | <0.0001 |
| CAGE | Q8TC20 | <0.0001 |

### Example 2

The inventor has further tested the applicability of the combination of the 8 autoantibodies, and analyzed the ability of the combination to distinguish between the patients with benign breast disease and the patients with triple-negative breast cancer.

The discovery cohort of 92 patients with benign breast disease (including benign breast mass, ductal ectasia, breast calcification, hyperplasia, etc.) and 40 patients with triple-negative breast cancer was analyzed.

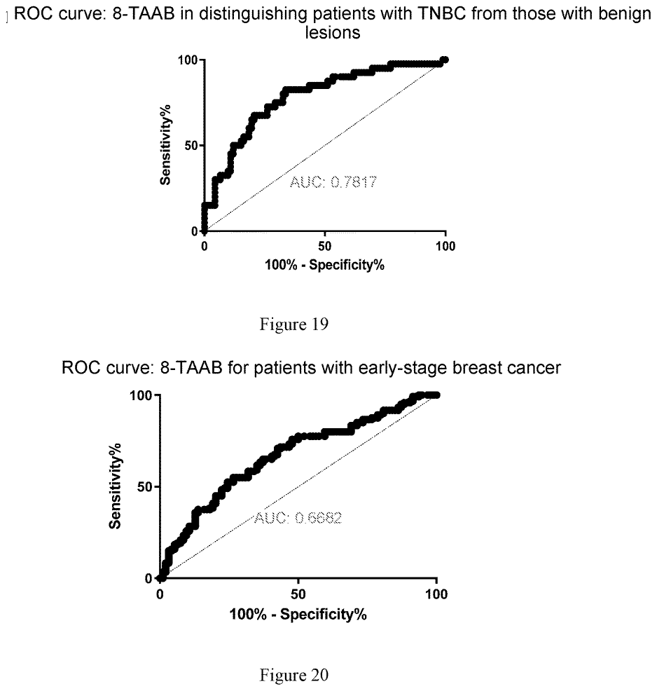

It can be seen from the receiver operating characteristic curve (FIG. 19) that the combination still well distinguished the patients with triple-negative breast cancer from the patients with benign breast disease with a sensitivity improved to 67.5%, a specificity remaining at 79.4%, a detection efficacy (AUC) of 0.7817, 95% CI (0.6956, 0.8678), and P<0.0001. It was thus demonstrated that the detection ability of the combination for triple-negative breast cancer was not disturbed by the benign breast diseases.

### Example 3

Except triple-negative breast cancer, the inventor continued to evaluate the detection ability of the 8-molecule autoantibody combination for early-stage breast cancer.

Another independent cohort including 100 patients with breast cancer (9 cases with ductal carcinoma in situ, 87 cases with invasive ductal carcinoma, 1 case with invasive micropapillary carcinoma, 1 case with invasive lobular carcinoma, and 2 cases with mucinous carcinoma) and 94 age-matched control subjects involved in this example. The 100 patients with breast cancer were all with stage I or II breast cancer according to TNM classification. Patient characteristics are shown in Table 4.

TABLE 4
Clinical characteristics of patients with early-stage breast cancer

| | Ductal | Invasive |
|---|---|---|
| | carcinoma | breast |
| | in situ | cancer |
| Number | 9 | 91 |
| % female | 100% | 100% |
| Average age | 49.9 ± 12.5 | 52.7 ± 11.1 |
| Age range | 31-67 | 27-77 |
| Pathological subtypes: Invasive ductal | | 87 |
| carcinoma | | |
| Invasive micropapillary carcinoma | | 1 |
| Invasive lobular carcinoma | | 1 |
| Mucosecretory carcinoma | | 2 |
| Lymphatic metastasis: Lymphatic metastasis | 0 | 10 |
| No lymphatic metastasis | 9 | 81 |
| ER expression state: ER (—) | 4 | 27 |
| ER (medium) | 1 | 4 |
| ER (high) | 4 | 60 |
| PR expression state: PR (—) | 4 | 38 |
| PR (medium) | 2 | 8 |
| PR (high) | 3 | 45 |
| HER2 expression state: HER2 (0) | 0 | 21 |
| HER2 (1+) | 1 | 38 |
| HER2 (2+) | 1 | 11 |
| HER2 (3+) | 7 | 21 |
| Triple-negative breast cancer or not: Yes | 0 | 12 |
| No | 9 | 79 |

The receiver operating characteristic curve (ROC) of the autoantibody combination of the present disclosure showed that the combination had a sensitivity of 45%, a specificity of 79.8%, a detection efficacy (AUC) of 0.6682, 95% CI (0.5957, 0.7408), and P<0.0001 for early-stage (stage I or II) breast cancer as shown in FIG. 20. Although the sensitivity of the combination was slightly decreased for early breast cancer compared with that for triple-negative breast cancer, the combination may still retain an inherent specific advantage in early detection of breast cancer.

According to the different subtypes of these patients, the sensitivity for early-stage ductal carcinoma in situ (DCIS, 9 cases) was 43%; the detection sensitivity for early-stage invasive ductal carcinoma (IDC, 87 cases) in the cohort was 46%. The results are shown in Table 5. Therefore, it may also be deduced that the 8-molecule autoantibody combination may be universally used in detection of different subtypes of early-stage breast cancer.

TABLE 5
Detection sensitivity of autoantibody combination
for different subtypes of early-stage breast cancer

| Cancer | Number of | |
|---|---|---|
| subtype | patients | Sensitivity |
| DCIS | 9 | 43% |
| IDC | 87 | 46% |

All the patents and publications mentioned in the description of the present disclosure indicate that these are public technologies in the art and may be used by the present disclosure. All the patents and publications cited herein are listed in the references, just as each publication is specifically referenced separately. The present disclosure described herein may be realized in the absence of any one element or multiple elements, one restriction or multiple restrictions, where the limitation is not specifically described here. For example, the terms “comprising”, “essentially consisting of”, and “consisting of” in each example herein may be replaced by the rest 2 terms. The so-called “a” here only means “a kind”, not excluding only one, but also may indicate two or more. The terms and expressions used herein are descriptive, without limitation. Besides, there is no intention to indicate that these terms and interpretations described in the description exclude any equivalent features. However, it can be known that any appropriate changes or modifications may be made within the scope of the present disclosure and claims. It can be understood that the examples described in the present disclosure are some preferred examples and features. A person skilled in the art may make some modifications and changes according to the essence of the description of the present disclosure. These modifications and changes are also considered to fall within the scope of the present disclosure and the scope limited by independent claims and dependent claims.

1. A method for detecting whether an individual suffers from a breast cancer, comprising the following steps:
providing a biological sample for an individual; and
detecting an autoantibody of an antigen protein in the biological sample to obtain a quantitative value of the autoantibody,
wherein if the quantitative value of the autoantibody exceeds or equals to a cutoff value, the individual is determined to be a patient with a breast cancer; if the quantitative value of the autoantibody is less than the cutoff value, the individual is determined not to be a patient with a breast cancer; and the antigen is selected from at least one or more than one antigen combination of the following antigens: PARP1, TRIM21, P53, BRCA2, Annexin 11, ATAD2, NY-ESO-1, and CAGE.

2. The method according to claim 1, wherein the antigen is PARP1, TRIM21, and CAGE.

3. The method according to claim 1, wherein the antigen is selected from one of the antigen combination:
(1) PARP1, TRIM21, and CAGE;
(2) PARP1, TRIM21, CAGE, and P53;
(3) PARP1, TRIM21, CAGE, and Annexin 11;
(4) PARP1, TRIM21, CAGE, and ATAD2;
(5) PARP1, TRIM21, CAGE, and NY-ESO-1;
(6) PARP1, TRIM21, CAGE, and BRCA2;
(7) PARP1, TRIM21, CAGE, Annexin 11, and ATAD2;
(8) PARP1, TRIM21, CAGE, Annexin 11, and NY-ESO-1;
(9) PARP1, TRIM21, CAGE, Annexin 11, and P53;
(10) PARP1, TRIM21, CAGE, ATAD2, and NY-ESO-1;
(11) PARP1, TRIM21, CAGE, ATAD2, and P53;
(12) PARP1, TRIM21, CAGE, NY-ESO-1, and P53;
(13) PARP1, TRIM21, CAGE, Annexin 11, ATAD2, and NY-ESO-1;
(14) PARP1, TRIM21, CAGE, Annexin 11, NY-ESO-1, and P53;
(15) PARP1, TRIM21, CAGE, ATAD2, NY-ESO-1, and P53; or
(16) PARP1, TRIM21, P53, BRCA2, Annexin 11, ATAD2, NY-ESO-1, and CAGE.

4. The method according to claim 1, wherein the sample of the individual is selected from one or more of the following samples: whole blood, serum, plasma, a tissue, a cell, interstitial fluid, cerebrospinal fluid, or urine.

5. The method according to claim 1, wherein the individual is a mammal.

6. The method according to claim 1, wherein the autoantibody is IgA, IgM, or IgG.

7. The method according to claim 1, wherein the breast cancer comprises a ductal carcinoma in situ and an invasive breast cancer; or a triple-negative breast cancer and a non-triple-negative breast cancer, or a stage I or II breast cancer.

8. The method according to claim 1, wherein the detection comprises one of the following modes: an enzyme-linked immunosorbent assay (ELISA), a protein/peptide fragment chip detection, an immunoblotting, a microbead immunoassay or a microfluidic immunoassay.

9. The method according to claim 1, wherein the absorbance cutoff value of the autoantibody of the antigen is as follows: Anti-PARP1 has an absorbance cutoff value of 1.5, Anti-TRIM21 has an absorbance cutoff value of 0.5, Anti-P53 has an absorbance cutoff value of 1.2, Anti-BRCA2 has an absorbance cutoff value of 1.0, Anti-Annexin 11 has an absorbance cutoff value of 1.0, Anti-ATAD2 has an absorbance cutoff value of 0.5, Anti-NY-ESO-1 has an absorbance cutoff value of 1.0, and Anti-CAGE has an absorbance cutoff value of 1.1.

10. A method for detecting whether an individual suffers from a triple-negative breast cancer, comprising the following steps:
providing a biological sample for an individual; and
detecting an autoantibody of an antigen protein in the biological sample to obtain a quantitative value of the autoantibody,
wherein if the quantitative value of the detected autoantibody of the antigen exceeds or equals to a cutoff value, the individual is determined to be a patient with a triple-negative breast cancer; if the quantitative value of the detected autoantibody of the antigen is less than the cutoff value, the individual is determined not to be a patient with a breast cancer; and the antigen is selected from at least one or more than one antigen of the following antigens: PARP1, TRIM21, P53, BRCA2, Annexin 11, ATAD2, NY-ESO-1, and CAGE.

11. The method according to claim 10, wherein the antigen is PARP1, TRIM21, and CAGE.

12. The method according to claim 10, wherein the antigen is selected from one of the antigen combination:
(1) PARP1, TRIM21, and CAGE;
(2) PARP1, TRIM21, CAGE, and P53;
(3) PARP1, TRIM21, CAGE, and Annexin 11;
(4) PARP1, TRIM21, CAGE, and ATAD2;
(5) PARP1, TRIM21, CAGE, and NY-ESO-1;
(6) PARP1, TRIM21, CAGE, and BRCA2;
(7) PARP1, TRIM21, CAGE, Annexin 11, and ATAD2;
(8) PARP1, TRIM21, CAGE, Annexin 11, and NY-ESO-1;
(9) PARP1, TRIM21, CAGE, Annexin 11, and P53;
(10) PARP1, TRIM21, CAGE, ATAD2, and NY-ESO-1;
(11) PARP1, TRIM21, CAGE, ATAD2, and P53;
(12) PARP1, TRIM21, CAGE, NY-ESO-1, and P53;
(13) PARP1, TRIM21, CAGE, Annexin 11, ATAD2, and NY-ESO-1;
(14) PARP1, TRIM21, CAGE, Annexin 11, NY-ESO-1, and P53;
(15) PARP1, TRIM21, CAGE, ATAD2, NY-ESO-1, and P53; or
(16) PARP1, TRIM21, P53, BRCA2, Annexin 11, ATAD2, NY-ESO-1, and CAGE.

13. The method according to claim 10, wherein the sample of the individual is selected from one or more of the following samples: whole blood, serum, plasma, a tissue, a cell, interstitial fluid, cerebrospinal fluid, or urine.

14. The method according to claim 10, wherein the individual is a mammal.

15. The method according to claim 10, wherein the autoantibody is IgA, IgM, or IgG.

16. The method according to claim 10, wherein the detection comprises one of the following modes:
an enzyme-linked immunosorbent assay (ELISA), a protein/peptide fragment chip detection, an immunoblotting, a microbead immunoassay or a microfluidic immunoassay.

17. The method according to claim 10, wherein the absorbance cutoff value of the autoantibody of the antigen is as follows: Anti-PARP1 has an absorbance cutoff value of 1.5, Anti-TRIM21 has an absorbance cutoff value of 0.5, Anti-P53 has an absorbance cutoff value of 1.2, Anti-BRCA2 has an absorbance cutoff value of 1.0, Anti-Annexin 11 has an absorbance cutoff value of 1.0, Anti-ATAD2 has an absorbance cutoff value of 0.5, Anti-NY-ESO-1 has an absorbance cutoff value of 1.0, and Anti-CAGE has an absorbance cutoff value of 1.1.

18. A method for detecting whether an individual suffers from a stage I or II breast cancer, comprising the following steps:
providing a biological sample for an individual; and
detecting an autoantibody of an antigen protein in the biological sample to obtain a quantitative value of the autoantibody,
wherein if the quantitative value of the detected autoantibody of the antigen exceeds or equals to a cutoff value, the individual is determined to be a patient with a stage I or II breast cancer; if the quantitative value of the detected autoantibody of the antigen is less than the cutoff value, the individual is determined not to be a patient with a breast cancer; and the antigen is selected from at least one or more than one antigen combination of the following antigens: PARP1, TRIM21, P53, BRCA2, Annexin 11, ATAD2, NY-ESO-1, and CAGE.

19. The method according to claim 18, wherein the antigen is PARP1, TRIM21, and CAGE.

20. The method according to claim 18, wherein the absorbance cutoff value of the autoantibody of the antigen is as follows: Anti-PARP1 has an absorbance cutoff value of 1.5, Anti-TRIM21 has an absorbance cutoff value of 0.5, Anti-P53 has an absorbance cutoff value of 1.2, Anti-BRCA2 has an absorbance cutoff value of 1.0, Anti-Annexin 11 has an absorbance cutoff value of 1.0, Anti-ATAD2 has an absorbance cutoff value of 0.5, Anti-NY-ESO-1 has an absorbance cutoff value of 1.0, and Anti-CAGE has an absorbance cutoff value of 1.1.
